(19) United States
(12) Patent Application Publication
(10) Pub. No.: US 2007/0110815 A1
(43) Pub. Date: May 17, 2007

(54) Micronised azodicarbonamide, and the preparation and use thereof

(76) Inventors: Michel Vandevelde, Bierges (BE); Helene Margery, Bierges (BE)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 11/586,723

(22) Filed: Oct. 26, 2006

(30) Foreign Application Priority Data
Oct. 26, 2005 (EP) 05110022.0

(51) Int. Cl. A61K 9/14; A61K 31/655

(52) U.S. Cl. 424/489; 514/150

(57) ABSTRACT

Azodicarbonamide (ADA) in the form of a micronised dry powder, said powder having a granulometric distribution of particles wherein the particles of the powder have a mean diameter (d₅₀) equal to or less than 2 μm and a 90% diameter (d₉₀) equal to or less than 4 μm.

The present invention relates to azodicarbonamide (ADA) in the form of a micronised dry powder, and to the preparation and use thereof.

Azodicarbonamide (ADA) has been known for a long time (1892) in the form of a crystalline chemical substance (the Merck Index, 11^{th} edition, 1989, p 938). This substance is not very soluble in water or in the majority of organic solvents, with the exception of N,N-dimethylformamide and dimethyl sulfoxide.

ADA has the general chemical formula NH₂—CO—N═N—CO—NH₂.

ADA also means, within the meaning of the present invention, each of the cis and trans isomers of this substance as well as the racemic mixtures thereof.

This substance is used as a swelling agent in the rubber and plastic industries. At a temperature of approximately 190°-230° C. azodicarbonamide decomposes into gases (nitrogen, carbon monoxide, carbon dioxide and ammonia), into solid residues and into subliminated substances.

It has also been used for improving flours in baking.

Some time ago, it was discovered that ADA also had a therapeutic effect against various disorders, in particular viral infections, certain cancerous diseases and disorders resulting from a pathological production of cytokines (see EP-B-0524961, EP-B-0941098, EP-B-1032401 and U.S. Pat. No. 5,585,367).

In order to carry out tests on animals and on human beings, it has proved necessary to improve the speed of dissolution of ADA and its bio-availability in blood and it has therefore been attempted to carry out a micronisation of this substance.

A product Celogen® AZ-2990, which is put on the market by the company Uniroyal, is known. This product is comprised of a mixture of micronised ADA and of an inert flow-conditioning agent which renders this product contra-indicated in the pharmaceutical field. If the nominal size of the particles of this mixture is cited as being 2-2,4 microns, the granulometric distribution of the particles is unknown.

In the US-2005/0222281A1, the possibility of producing micronised ADA by means of an air jet disintegrator is evoked. This document advises against such a process; it states that, on one hand, if this process was to be exploited, it would be theoretically uneconomic because involving an enormous energy consumption and, on another hand, the so obtained powders would show a wide distribution of the particle sizes, with great flow problems for the so obtained powder.

Methods of manufacturing very pure ADA have been known for a long time, and even methods for achieving particle sizes on a micron scale (see for example GB-1181729).

However, the micronisation obtained is insufficient since the granulometric distribution of the particles is very wide, which impairs the reproducibility of the results that could be obtained with this product, if it were applied for example in pharmacy.

A homogeneous micronisation of ADA has proved to be very difficult to achieve, since this is an extremely hard substance.

Several tests have been attempted for this purpose. According to WO-01/03670 a micronisation of ADA in an aqueous dispersion medium has proved to not be very advantageous since it gives rise to a foam which remains stable for several weeks. Thus it is recommended in this document to proceed rather with a micronisation in a non-aqueous liquid medium, at high pressure (500-700 bar). Non-aqueous means, in WO-01/03670, an organic liquid, for example polyethylene glycol 400, to which there is added, in order to improve dispersion, a surfactant such as Tween 80.

According to the teachings of this document, it is thus possible to obtain ADA having a d₅₀ of 3.0 μm to 5.5 μm, certain particles of which can achieve up to more than 7.0 μm. This product does of course still contain traces of the dispersion medium. Polyethylene glycol is a pharmaceutically toxic substance which it is necessary to eliminate by degassing at high temperature.

This process is therefore complex and expensive. It scarcely improves the micronisation obtained by adjustment of the chemical conditions of the reaction described in GB-1181729 and in general there is a risk of its spoiling the ADA by the use of high pressures and temperatures. Moreover, the final product obtained cannot have a degree of pharmaceutical purity and, given its wide granulometric distribution, there is a risk that it may not meet the requirements of reproducibility as a pharmaceutically active substance.

In summary, ADA produced according to the technical background is under the form of very hard crystals which are difficult to micronise, while avoiding degradation of ADA during micronisation and a back aggregation of the micronised fine particles after micronisation. Up to now, in order to obviate these drawbacks, the provided solutions were an unsatisfying micronisation in wet medium or an addition of surfactants to the micronised ADA or a coating of the ADA particles, what renders the product unusable in the pharmaceutical field.

The aim of the present invention is to resolve the problems posed by proposing an azodicarbonamide having good bio-availability properties, which, at therapeutically active doses, has drastically reduced if not zero toxicity. In addition, it may be important and desirable to have an ADA where the particle size increases the reproducibility of the characteristics of the active substance, as required by the Administrations that authorise the marketing of pharmaceutical substances. Finally, the ADA particle size must advantageously be varying as little as possible, during storage.

These problems are resolved according to the invention by azodicarbonamide (ADA) in the form of a micronised dry powder, which is characterised by the fact that the said powder has a granulometric distribution of particles wherein the particles of the powder have a mean diameter (d₅₀) equal to or less than 2 μm, preferably equal to or less than 1.8 μm, advantageously around 1.5-1.6 μm. The particles of the powder have also a 90% diameter (d₉₀) equal to or less than 4 μm, advantageously around 3.4 to 3.9 μm. In a particularly preferable manner, the ADA has a degree of pharmaceutical purity in particular greater than 98%, especially greater than 98.4%. ADA is advantageously free of surfactant or of any other additive provided for empeding an aggregation of the fine particles. The ADA particles are not covered with a coating. Preferentially the ADA micronised particles according to the invention have a 10% diameter (d₁₀) equal to or less than 0.6 μm.

In order to obtain an ADA having such a size fineness and simultaneously such a narrow granulometric distribution, which have up till now been impossible to achieve, there has been provided, according to the invention, a method comprising the steps of
oxidizing biurea in suspension in water by chlorine gas or hydrogen peroxide, at ambient temperature and pressure,
separating azodicarbonamide, by filtration,
washing and thereafter drying azodicarbonamide,
air jet disintegrating azodicarbonamide in the dry state, at a pressure lower than 100 bar, with formation of micronised particles, and
selecting micronised particles having a size lower than a value of 5 μm.

Such a method offers the advantage of not diluting or contaminating the ADA in other substances that are subsequently undesirable and being able to proceed at moderate pressures and temperatures that do not risk spoiling the ADA. Preferably the step of producing azodicarbonamide is carried out in pressure and temperature conditions which are ambient or close thereto. In the same manner, the step of drying the produced ADA takes place in moderate pressure and temperature conditions, for example the ambience. Applying the above mentioned moderate conditions during the ADA production has also as unexpected result that the ADA crystals to disintegrate are clearly less hard that the crystals obtained according to the technical background, what allows the use of moderate temperature and pressure during the disintegration. Advantageously, the pressure in the air jet disintegrator is below 100 bar, and preferably below 70 bar, in particular around 60 bar. The disintegration in an air jet preferably takes place at a temperature below a decomposition temperature of ADA (190-230° C.), advantageously at ambient temperature. The micronised ADA does not undergo any spoiling or only a little during the disintegration, and, as discussed subsequently, it was possible to observe the formation of a powder, the particle size thereof remains stable during very long periods, without additive.

The present invention also relates to a pharmaceutical composition containing, as an active substance, ADA in the form of a dry powder micronised according to the invention. These compositions may contain a pharmaceutically compatible excipient and one or more adjuvants normal in pharmacy. It is also possible to envisage compositions according to the invention containing ADA and at least one other therapeutically active substance in association, such as for example AZT, ritonavir, T-20 or the like.

Such a composition may for example be in the form of a powder, a tablet, a pill, a capsule, a sugar-coated pill, a suspension, a cream, a paste, a syrup or sachets. The composition may be administered in a normal manner, for example by an oral, sublingual, rectal, vaginal, local, transcutaneous or transmucous method or by injection or perfusion.

The present invention also concerns a method of preparing a pharmaceutical composition as indicated above, this method comprising associating ADA according to the invention with a pharmaceutically compatible excipient, as well as normal adjuvants.

The present invention also concerns a use of ADA according to the invention for manufacturing a medicament to be used in the treatment of viral illnesses, in particular infections by viruses containing a protein of the so-called “zinc finger” type. This would mean in particular the treatment of human or animal infections by papilloma viruses, retroviruses, in particular the human immunodeficiencyvirus, arenaviruses, herpes viruses and the hepatitis C virus.

The use of ADA according to the invention is also envisaged for manufacturing a medicament to be used in the treatment of human or animal ailments resulting from a pathological production of cytokines or lymphokines as well as for the manufacture of a medicament to be used in the treatment of human or animal ailments giving rise to a high pathological cellular production of deoxyribonucleic acid, of the cancerous type.

The present invention also concerns a use of ADA according to the invention and a pharmaceutical composition containing ADA according to the invention for the treatment of cells of microorganisms, isolated cells, macroorganisms and cells of an organism or cellular tissue extracted from a human or animal body, in particular a graft.

The invention will now be described in more detail with the help of non-limiting examples.

### BRIEF DESCRIPTION OF THE DRAWINGS

FIG. 1 shows a histogram in which the values on the X-axis are the administrated doses of ADA and on the Y-axis the concentrations of biourea in the blood plasma,

FIG. 2 shows the size distribution of ADA crystals after manufacture according to the invention, and

FIG. 3 shows the stability of ADA manufactured according to the invention.

### EXAMPLE 1

### Method of Preparing ADA

In a known manner, hydrazine sulphate and urea are made to react in order to form biurea, also referred to as hydrazodicarbonamide. The water is used as a solvent. Biurea is then made to precipitate, is filtered and washed with water.

The biurea is then put in suspension in water and a mixture of chlorine gas and an inert gas, optionally in presence of an inert gas, such as air, nitrogen or carbon dioxide, is bubbled in this medium, which causes an oxidation of the biurea with the formation of a double bond between the central nitrogens, which gives azodicarbonamide. This product is next filtered, washed with water until conditions close to neutrality are obtained and dried at a temperature close to the ambient temperature, preferably to that temperature.

Azodicarbonamide is sensitive to high pressures and temperatures, since it then forms by-products such as semicarbazide, hydrazine and/or biurea, and the formed ADA crystals are characterized by an exceptional hardness.

Using an HPLC method it was possible to determine the obtaining of azodicarbonamide produced according to the process of this example to a degree of purity above 98%, in particular 98.4%, that is to say pharmaceutical quality.

### EXAMPLE 2

### Method of Micronising ADA According to the Invention

Three batches of ADA manufactured according to the method of example 1 are used, each batch weighing 15 kg.

A thorough fragmentation of dry powder forming each of these batches is carried out, feeding them at a rate of 4 kg/h into a known dry powder disintegration device, for example into a device of the Alpine® 100 AFG Jet Mill model from Hosokawa Micron Group. This device comprises a cylindrical milling chamber with a conical bottom which is made of stainless steel coated with an elastomer. This chamber has a diameter of 100 mm and a volume of 800 cm³. The particles to disintegrate are introduced into the chamber by an endless screw. Then the particles are projected against each other by compressed air jets from 3 air nozzles having a diameter of 2 mm which run into each other at the same point. The obtained air stream carries then the disintegrated particles at 50 bar towards a turbo-selecting device which is integrated within the milling chamber. In this example this turbo-selecting device has the shape of a squirrel-cage which has adjustable rotational speed of 5000 to 16000 rpm.

This device allows the exit of the particles smaller than a determined size, in this case <5 μm, and repulses into the milling chamber the particles having a greater size. The fine particles (searched size) which leave the milling chamber are collected for example by means of a cyclone.

Air is filtered before being returned to the atmosphere. It became clear that, using such a device, it was possible, at a moderate pressure of around 75 bar at the feed and 60 bar inside, to micronise the ADA in a single three-hour cycle.

The sizes of the particles were then analysed. In order to analyse the diameters use was made of a RODOS & RODOS/M dry powder dispersion device (from Sympatec GmbH) and a VIBRI vibratory powder feed tool (from Sympatec GmbH) with a HELOS laser diffraction system (from Sympatec GmbH). The powders analysed were fed at a rate of 35% of the maximum rate. On passing through an air jet, the powder is subjected to shearing forces of around 3.0 bar. Using a vacuum (90-100 mbar) they are then sucked into the path of a HE/NE laser beam. The diffraction of the laser beam by the particles creates a model which is measured and converted by computer into a particle size distribution using software associated with the HELOS system.

| Batches | | |
|---|---|---|
| A | B | C |

| | | | | | | |
|---|---|---|---|---|---|---|
| Feed | 8.5 | bar | 8.5 | bar | 8.1 | bar |
| pressure to | | | | | | |
| disperser | | | | | | |
| Pressure | 8.0 | bar | 7.8-8.1 | bar | 8.0-8.1 | bar |
| inside | | | | | | |
| disperser | | | | | | |
| Feed rate | 25-26 | g/min | 26-27 | g/min | 26-27 | g/min |

| | Granulometry of particles during micronisation (μm) | | | | | | | | |
|---|---|---|---|---|---|---|---|---|---|
| | d₁₀ | d₅₀ | d₉₀ | d₁₀ | d₅₀ | d₉₀ | d₁₀ | d₅₀ | d₉₀ |
| Start | 0.58 | 1.61 | 3.78 | 0.58 | 1.58 | 3.71 | 0.56 | 1.47 | 3.43 |
| Middle | 0.56 | 1.53 | 3.55 | 0.58 | 1.59 | 3.88 | 0.57 | 1.56 | 3.69 |
| End | 0.56 | 1.51 | 3.50 | 0.59 | 1.56 | 3.68 | 0.57 | 1.58 | 3.85 |

| | | | |
|---|---|---|---|
| Efficiency | 97.8 | 100.0 | 98.4 |
| of disinte- | | | |
| gration (%) | | | |

### EXAMPLE 3

### Comparative Examination of Bioavailability

54 mice are randomly divided into 9 groups of 6 animals and receive a dose of ADA by force feeding.

Group A receives 10 mg of micronised ADA according to the invention (in suspension in carboxymethylcellulose (CMC)) per kg of bodyweight.

Group B receives 10 mg/kg of non-micronised ADA (in suspension in CMC).

Group C receives 10 mg/kg of ADA in solution in dimethyl sulfoxide (DMSO).

Group D receives 5 mg/kg of micronised ADA according to the invention (in suspension in CMC).

Group E receives 5 mg/kg of non-micronised ADA (in suspension in CMC).

Group F receives 5 mg/kg of ADA in solution in DMSO.

Group G receives 1.25 mg/kg of micronised ADA according to the invention (in suspension in CMC).

Group H receives 1.25 mg/kg of non-micronised ADA (in suspension in CMC).

Group I receives 1.25 mg/kg of ADA in solution in DMSO.

The concentration of biurea, the only catabolite of ADA “in vivo” (Concise International Chemical Assessment Document 16; World Health Organisation: Geneva, 1999) in the blood serum (μg/ml) was determined 30 minutes after ingestion by means of a high-pressure liquid chromatography method. This concentration represents a measurement of the bioavailability of ADA in the organism.

Analysis of the plasma samples was carried out by high-pressure liquid chromatography, followed by mass spectrometry, in accordance with the standards of the “Food and Drug (USA) Administration May 2001: Guidance for Industry: Bioanalytical method validation” (lowest quantification limit (LOQ) of 0.20 μg/ml and linear method up to 20 μg/ml).

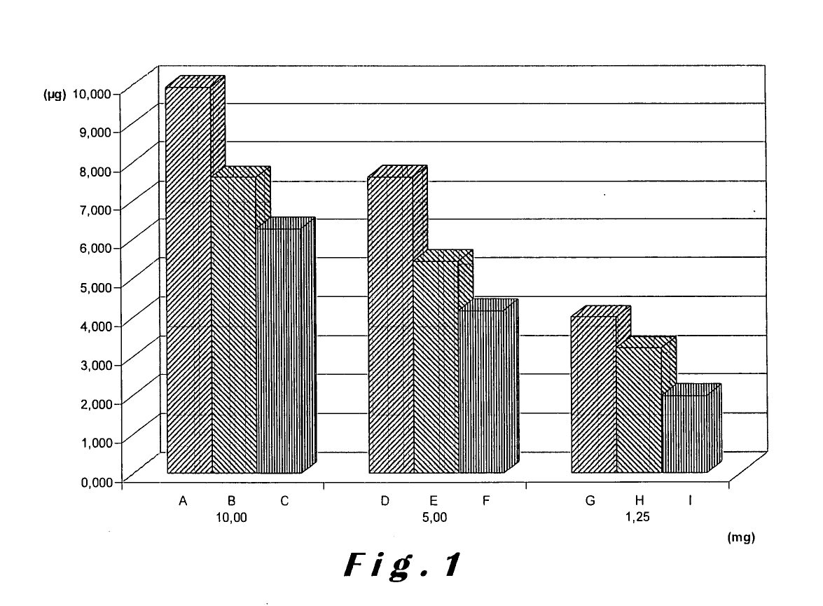

The results obtained are indicated in the appended FIG. 1, which shows a histogram in which the values on the X-axis are the doses of ADA administered in mg/kg of body weight and the values on the Y-axis the concentrations of biourea in μg/ml in the blood plasma.

As can be seen, the groups that received micronised ADA according to the invention (groups A, D and G) have at all doses a bioavailability of ADA in the blood appreciably better than the non-micronised ADA.

Surprisingly, the ADA according to the invention has even better bioavailability than completely dissolved ADA.

### EXAMPLE 4

### Examination of Toxicity on Animals

The toxicity of azodicarbonamide administered orally is examined. It is known that, when ADA as commercially available and having a d₅₀ of 18 μm and a d₉₀<35 μm is administered, the formation of biurea crystals is quickly observed in the urine.

Tests were carried out on rats to which daily doses of 900, 150 and 25 mg of ADA according to the invention/kg of body weight were administered orally for 28 days. The ADA was in the form of a micronised powder according to the invention in suspension in carboxymethylcellulose. Likewise an identical test was carried out on dogs at daily doses for 28 days of 400, 100 and 25 mg of ADA according to the invention/kg of body weight. In neither of the two tests was any adverse effect observed.

On the dogs that received 400 mg/kg/day for 28 days, a histological examination was also carried out with dissection of the kidneys. Entirely surprisingly no biurea crystals were observed.

Given the better bioavailability obtained in the blood, an increased formation of crystals of the metabolite of ADA might on the contrary have been expected, at such doses, which proved not to be the case.

### EXAMPLE 5

### Examination of Toxicity on Human Beings

“ESB free” gelatine-coated capsules containing a composition as described in example 4 were administered for seven days to healthy male volunteers at a dose ranging up to 6 g per day (single dose of 150 to 6,000 mg and repeated doses of 300 to 2,400 mg).

A urine examination was carried out using flow cytometry (limit of detection 2 μm). No kidney stones or cylinders were observed.

### EXAMPLE 6

### Examination of Reactivity of Respiratory Tracts

At day 0, Brown Norway rats (200-250 g) were sensitised by intraperitoneal administration (1 ml/rat) of ovalbumin (1 mg/ml) mixed with aluminium hydroxide (100 mg/ml).

In this experiment, micronised ADA according to the invention is administered to rats by force feeding in the form of a suspension in poloxamer 188. The administration takes place twice a day in doses of 125 and 250 mg/kg, from day −1 up to day 20.

It was noted that this treatment with ADA gives rise to a significant inhibition of the increase obtained for the control animals in the reactivity of the respiratory tracts to interleukins, at all doses of methacholine caused by antigen attack in sensitised animals as described above.

### EXAMPLE 7

### Composition of Capsule

In a capsule of the Yvory No 2 type, the following composition was introduced:

| | | |
|---|---|---|
| Azodicarbonamide according to the invention | 150 | mg |
| Glycerol monostearate (geleol) | 3 | mg |
| Colloidal anhydrous silica | 3 | mg |
| Vegetable magnesium stearate | 1 | mg |
| Isopropyl alcohol | 15 | mg (evaporated) |

### EXAMPLE 8

### Composition of Suspension to be Introduced in a Brown-glass Flask Protecting vis-à-vis Light

| | | |
|---|---|---|
| Azodicarbonamide according to the invention | 1.00 | g |
| PVP | 0.20 | g |
| PF68 | 0.20 | g |
| Distilled water | 98.60 | g |

This suspension can be used in paediatrics (1 ml of suspension gives 1 mg of ADA, the expected dose being 10 mg/kg of body weight, twice per day).

### EXAMPLE 9

### Composition of Vaginal Cream

This composition contains 50 mg of azodicarbonamide according to the invention as well as cetyl ester wax, cetyl alcohol, white wax, glyceryl monostearate, propylene glycol monostearate, methyl stearate, sodium lauryl sulphate, glycerine, mineral oil and benzyl alcohol as a preservative.

This microbicidal and virucidal composition may be useful for local and preventive usage.

### EXAMPLE 10

As previously indicated, one of the main difficulties associated with the ADA micronisation according to the technical background consists in the loss of the initial size by aggregation of the micronised particles into greater particles.

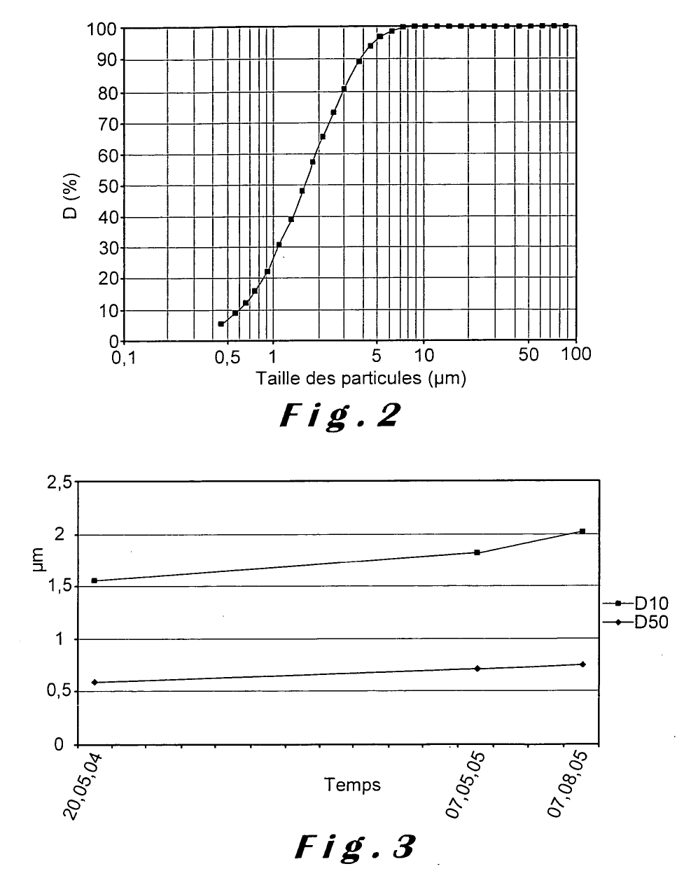

After having manufactured micronised ADA under the conditions of examples 1 and 2, a size distribution of the ADA crystals was obtained, as illustrated on the appended FIG. 2.

Thereafter the active substance was incorporated into capsules which have been stored for 11 months within a refrigerator at 8° C., in order to determine the stability of ADA according to the standards (International Harmonisation Conference: ICH).

The size of the ADA particles has been determined again at this moment as well as after 3 additional months within a stove at 25° C./60% humidity.

Particle sizes

| Code number | | | |
|---|---|---|---|
| of the batch | Test date | D10 | D50 |
| K9J | Jun. 20, 2004 | 0.56 μm | 1.51 μm |
| K9K | | 0.59 μm | 1.56 μm |
| K9L | | 0.57 μm | 1.58 μm |
| K9J | May 7, 2005 | 0.72 μm | 1.86 μm |
| K9K | | 0.69 μm | 1.74 μm |
| K9L | | 0.72 μm | 1.88 μm |
| K9J | Aug. 7, 2005 | 0.76 μm | 2.05 μm |
| K9K | 25° C./60% humidity | 0.74 μm | 1.93 μm |
| K9L | | 0.76 μm | 2.08 μm |

These results are reproduced on FIG. 3, from which the stability of ADA according to the invention clearly results, while no additive has been incorporated into the active substance.

It must be understood that the present invention is in no way limited to the embodiments described above and that many modifications can be made thereto without departing from the scope of the accompanying claims.

1. Azodicarbonamide (ADA) in the form of a micronised dry powder, said powder having a granulometric distribution of particles wherein the particles of the powder have a mean diameter (d₅₀) equal to or less than 2 μm and a 90% diameter (d₉₀) equal to or less than 4 μm.

2. Azodicarbonamide according to claim 1, wherein the particles of the powder have a 10% diameter (d₁₀) equal to or lower than 0.6 μm.

3. Azodicarbonamide according to claim 1, having a degree of purity greater than 98%.

4. Pharmaceutical composition containing, as a therapeutically active substance, azodicarbonamide according to claim 1.

5. Composition according to claim 4, containing, in addition to the azodicarbonamide, at least one other therapeutically active substance.

6. Composition according to claim 4, further containing a pharmaceutically compatible excipient and one or more adjuvants usual in pharmacy.

7. Composition according to claim 4, which is in the form of a powder, a tablet, a pill, a capsule, a sugar-coated pill, a suspension, a cream, a paste, a syrup or sachets.

8. Composition according to claim 4, to be administered by oral, sublingual, rectal, vaginal, local, transcutaneous or transmucus method or by injection or perfusion.

9. Method of preparing azodicarbonamide according to claim 1, comprising the steps of
oxidizing biurea in suspension in water by chlorine gas or hydrogen peroxide, at ambient temperature and pressure,
separating azodicarbonamide, by filtration,
washing and thereafter drying azodicarbonamide,
air jet disintegrating azodicarbonamide in the dry state, at a pressure lower than 100 bar, with formation of micronised particles, and
selecting micronised particles having a size lower than a value of 5 μm.

10. Method according to claim 9, wherein drying and/or disintegrating steps take place at the ambient temperature.

11. Method of preparing a pharmaceutical composition according to claim 4, comprising associating azodicarbonamide (ADA) in the form of a micronised dry powder, said powder having a granulometric distribution of particles wherein the particles of the powder have a mean diameter (d₅₀) equal to or less than 2 μm and a 90% diameter (d₉₀) equal to or less than 4 μm and at least one pharmaceutically compatible excipient.

12. A method of treating a human or other animal infected with a virus containing a so-called “zinc finger” protein, said method comprising the step of administering a therapeutically effective amount of azodicarbonamide according to claim 1.

13. A method of treating according to claim 12 for the treatment of human or animal infections by papilloma viruses, retroviruses, arenaviruses, herpes viruses and the hepatitis C virus.

14. A method of treating human or animal ailments resulting from a pathological production of cytokines or lymphokines, said method comprising the step of administering a therapeutically effective amount of azodicarbonamide according to claim 1.

15. A method of treating human or animal ailments giving rise to a high pathological cellular production of deoxyribonucleic acid of the cancerous type, said method comprising the step of administering a therapeutically effective amount of azodicarbonamide according to claim 1.

16. A method of treating cells of micro-organisms, isolated cells, macro-organisms and cells of an organism or cellular tissue extracted from a human or animal body, said method comprising the step of administering a therapeutically effective amount of azodicarbonamide according to claim 1.
